(19) United States
(12) Patent Application Publication
(10) Pub. No.: US 2022/0117714 A1
(43) Pub. Date: Apr. 21, 2022

(54) BIODEGRADABLE WRAP FOR VETERINARY USE, ESPECIALLY LEG WRAPS FOR CATTLE

(71) Applicant: Zinpro Corporation, Eden Prairie, MN (US)

(72) Inventors: Peter A. Stark, Inver Grove Heights, MN (US); Arturo Gomez Rivas, Santiago de Compostela (ES); Roger Olson, Hammond, WI (US)

(21) Appl. No.: 16/949,152

(22) Filed: Oct. 15, 2020

(51) Int. Cl. A61D 9/00; A61L 15/28; A61L 15/44; A61L 15/56; A61F 13/06

(52) U.S. Cl. CPC A61D 9/00; A61L 15/28; A61F 13/06; A61L 15/56; A61L 15/44

(57) ABSTRACT

A biodegradable, non-pressure compression leg wrap for cattle suffering lameness from for example digital dermatitis with disintegration time controlled by the ratio of polyvinyl alcohol to copolymer starch or cellulose in the wrap composition.

### FIELD OF THE INVENTION

The present invention relates to polyvinyl alcohol based polymer, preferably with starch copolymers and minors to make bandage wraps that are biodegradable. More specifically the present invention relates to polyvinyl alcohol polymers (PVA) plus starch copolymers, and/or other minors with the composition tailored to a certain time desired degradation property, all by control of the PVA to other copolymer ratio along with the amount of material. The composition can be made into a self-adhering biodegradable wrap, especially useful for treating lameness diseases in cattle, such as digital dermatitis.

### BACKGROUND OF THE INVENTION

Lameness is a severe problem in cattle and has a detrimental effect on their longevity, productivity, and reproductive performance. It has been reported as among the disorders causing the largest economic losses in the dairy industry (Enema and Ostergaard, 2006). The majority of the cases of leg lameness involve foot lesions.

Lameness is especially critical on dairy farms because of the environmental conditions, moistness, the pressure on the legs from standing, and the dirt, and manure, all combine to make an environment ready for lameness diseases such as digital dermatitis.

Digital dermatitis in dairy cattle has the capability of effecting health and milk production yield.

One of the common treatments for this painful hoof disease is application of antibiotics and closed bandage wraps which eventually must be removed following the treatment.

Left untreated, digital dermatitis leads to hoof lesions that ultimately result in open wounds. Treatments without a wrap application have shown only limited efficacy in the disease treatment and the prevention of relapses. However, the application of a wrap which does increase treatment contact time against the lesion means that the topical wrap itself must at some critical time be removed in order to allow open air circulation for healing. Eventually the used wrap has to be discarded.

Ordinarily topical treatments involving the use of wraps are meant to be removed in a few hours or at most a few days after treatment. When wraps stay on for longer than recommended given the frequent presence of manure and dirt in cows feet, the incidence and severity of the disease can increase by the contamination. Although the wrap may at any time be removed by farm operators given the complexity of managing animals individually, commonly wraps stay longer than prescribed, creating additional problems, often even greater than the original lesions.

The most common wraps used in the field are elastic and based on a self-attaching technology (no glue or clip). These types of pressure wraps, unless applied with specific care, can create too much pressure on the wounds. Constant wound pressure exacerbates the lesion problem.

Further, wraps which easily fall off of the wrapped hoof for example or lame leg have the risk of clogging farm equipment and potentially contaminating the environment.

In order to address the current limitations on wound dressing wraps, particularly for lameness of cattle, there is a particular need for a wound dressing having the following characteristics: a wrap which dissolves and/or degrades in a period of hours or days in a moist environment in order to allow proper contact time between the treatment and the wound. A wrap that does not need to be removed physically, facilitating the handling of the animals, and decreasing associated stress on the animal. A wrap that is not elastic, avoiding the risk of pressure wounds. A wrap that is biodegradable and therefore does not plug farm equipment and is therefore environmentally friendly.

The primary objective of the present invention is to develop a wrap formulation and wrap that fulfills the above particular needs, as well of others.

### SUMMARY OF THE INVENTION

The most important feature of the new wrap is that water is the natural solvent for it and the specific formulation guarantees a controlled minimum amount of contact time between the treatment and the skin and then the wrap degrades in a period between two hours and ten days at a rate controlled by the formulation itself, its thickness, and the number of wraps around the animal. Since moisture determines the rate at which the bandage degrades, in high moisture environments the bandage will dissolve at a faster rate.

The wrap composition is a polyvinyl alcohol polymer based composition preferably with a copolymer composition (preferably starch or cellulose copolymer), with its formulation tailored to disintegration times that are desirable for the cattle operation involved. Controlling the amount of copolymer and PVA can control the rate of degradation. The most preferred compositions are polyvinyl alcohol, from 50% to 75% by weight, copolymer 20% to 40% by weight and up to 20% minors by weight. When the formulation is controlled as herein mentioned, the bandage has integrity, and results in ease of application, is not a pressure point bandage, and it can be supplied in rolls of different thickness that can be customizable depending on the target use. For example, the targeted wrap can be supplied in widths ranging from 4 centimeters to 50 centimeters. The thickness can also be tailored with a practical range being between 20-200 microns with a preferred range being between 40 and 80 microns. Other minor additives and/or plasticizers, medicaments, bioactives, deodorants, disinfectants, colors, and others that do not affect bandage integrity.

The details of the composition and the method of making it will be apparent from the detailed description of the invention which follows herein after.

### BRIEF DESCRIPTION OF THE DRAWINGS

FIG. 1 shows the wounded skin of an animal suffering from digital dermatitis.

FIG. 2 shows the initial application of a treatment, with a gauze over the treated area ready for the bandaging wrap of this invention.

FIG. 3 shows the initial interdigital placement of the bandage tail.

FIG. 4 shows the initial wrap of the complete circumference of the foot.

FIG. 5 shows a right and left interdigital wrap bandage procedure.

FIG. 6 shows a repetition of wrapping procedure of FIGS. 4 and 5.

FIG. 7 shows the final end of the wrap of the bandage being secured to its tail end.

### DETAILED DESCRIPTION OF A PREFERRED EMBODIMENT

For making the bandage composition with a controlled rate of wrap disintegration, one adjusts the polyvinyl alcohol (PVA) ratio to other copolymer. Generally, it has been found that if of the total bandage composition weight from about 50% to 100% is PVA, the rate of dissolution can be controlled to within 2 hours to 10 days. The rate of disintegration increases with the amount of PVA. A preferred weight amount of PVA is 50% by weight to 75% by weight, with the copolymer when employed being 20% by weight to 40% by weight.

The balance beyond PVA, and copolymer, if desired is chemical and medicinal and integrity modifiers and colorant up to 20%, preferably 15% to 20% by weight.

The PVA polyvinyl alcohol is a well-known synthetic polymer that is water soluble, commonly used in film making and treating textiles. It has good mechanical properties and is biodegradable, and widely available.

Starch is among the most abundant and inexpensive biopolymers. Starch is found in plant tissues, such as leaves, stems, seeds, roots, and tubers. It is also found in certain algae and bacteria. Starch exists in semi-crystalline granules of different size, shape and morphology depending on its botanical source. Nevertheless, most starches are composed of two structurally distinct molecules: amylose, a linear or lightly branched (1→4)-linked α-glucopyranose, and amylopectin, a highly branched molecule of (1→4)-linked α-glucopyranose with α-(1→6) branch linkages. The amylose/amylopectin ratio in starches varies with botanical origin.

Starches included in aspects of the present invention can be any naturally occurring starch, synthetic and/or physically or chemically modified starch. Non-limiting examples of included starches are bean starch, corn starch with amylose content of about 50% such as corn starch available commercially as Gelose 50, unmodified high amylose corn starch which contains approximately 55% amylose such as corn starch available commercially as Hylon V, unmodified high amylose corn starch which contains approximately 70% amylose such as corn starch available commercially as Hylon VII and corn starch with amylose content of about 80% available such as corn starch available commercially as Gelose 80.

The PVA/copolymer matrix preferred is starch copolymer, but cellulosic copolymers can also be used. For example, cellulose derived from cotton or other natural fibers. Cellulose esters and ethers can be used.

A very wide range of products can be prepared using, for example, different cellulose ethers. They differ from each other with respect to type of substituents, substitution level, molecular weight (viscosity), and particle size. The most common types of cellulose ethers are: Hydroxypropyl methyl cellulose (HPMC), Hydroxypropyl cellulose (HPC), Hydroxyethyl cellulose (HEC), and Sodium carboxy methyl cellulose (Na-CMC).

Pure cellulose as such is insoluble in hot or cold water due to strong intramolecular hydrogen bonding. So cellulose is converted to cellulose esters or cellulose ethers derivatives which are water soluble. These water soluble cellulose derivatives are used in wide range of applications. Thus, modified cellulose derivatives enhance water retention capacity, pseudoplastic behavior, film forming properties, and complexation. The advantages of cellulose ethers are that they are biocompatible and hence can be used for pharmaceutical purposes. They are mainly used medicinally as binders, coating agents, emulsifying, stabilizing, agents, and tablet disintegrants.

Other materials which can be included, and here collect minors, without limitation bioactive compounds, pharmaceuticals, deodorants, colorants, clays, etc. Generally, these are at levels of 5% or less by weight but in total may be up to 20% of the wrap by weight, preferably 15-20% by weight of the wrap.

The composition of raw materials PVA and starch copolymer and other minor additive materials are dry mixed in a turbo mixer and then put into a granulator and made into grains, and then extruded into mother coils, which as desired, can then be extruded into rolls of tape of any desired width or thickness, for example from 4 cm width to 50 cm width and 20 to 200 microns thick. Extrusion temperature can be in a range between 200 and 200° C. Extrusion pressure can be around 20 MPa (197 ATM).

The final composition is self-attaching with moisture, is not strongly elastic and is thus not prone to pressure wounds, and dissolves at a controlled rate that will allow proper contact time between the treatment and the wound. The amount of PVA and material controls the rate of disintegration. The more PVA in relation to the balance, and the less over all material, the quicker the wrap disintegrates. Finally, since it is bio-degradable it will not plug farm equipment.

Texture, morphology, and rheology can all be controlled by formulation adjustments as desired, all within the ranges herein specified.

The rate of disintegration is controlled by the PVA polymer to copolymer ratio. The ratio of PVA to a copolymer can be from 6:1 to 1:1 for 2.0 hours to 10 days and from 3:1 to 1:1 for 1 day to 5 days.

The following examples are offered to illustrate the formulation, preparation and use of the biodegradable wraps of the present invention.

### EXAMPLES

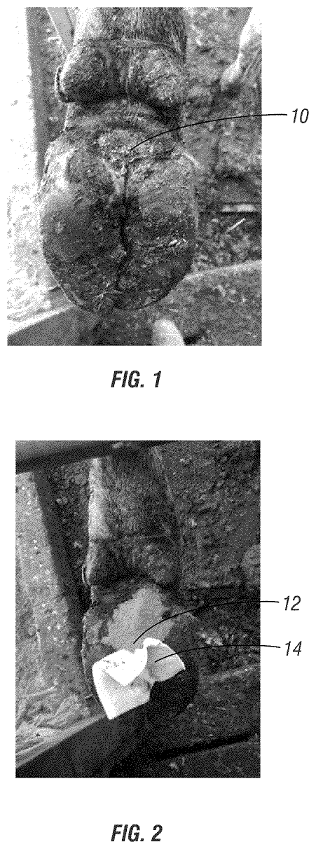

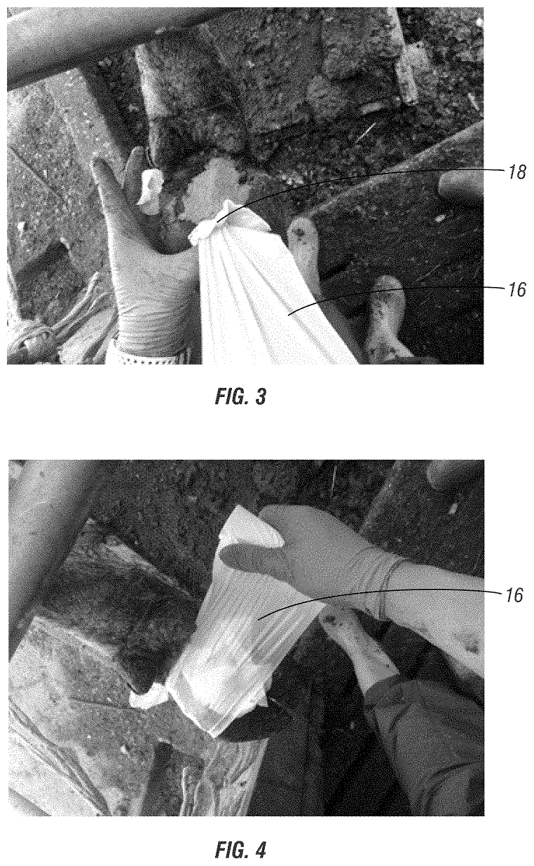

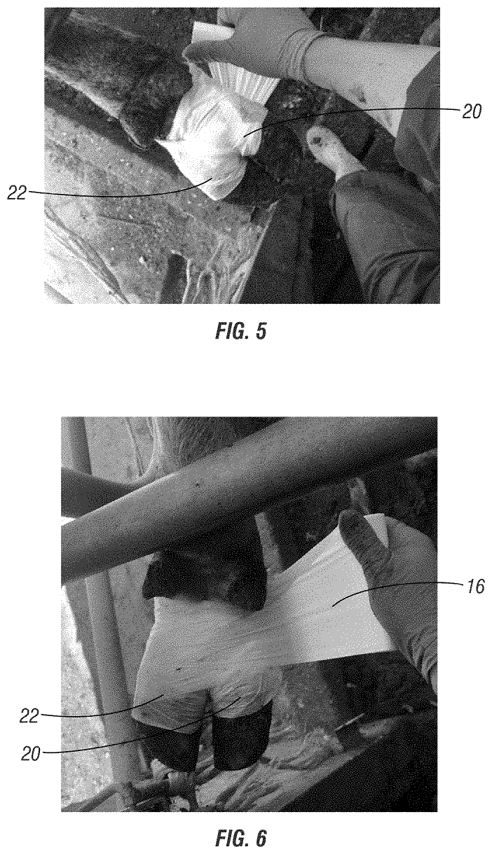

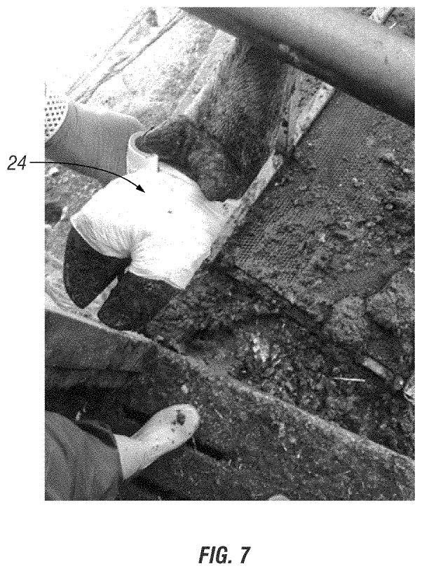

In each instance the wrap was tested after the wrapping procedure illustrated in FIGS. 1 through 7. FIG. 1 shows a cow's hoof of a cow suffering from digital dermatitis with the wound 10 clearly visible. FIG. 2 shows a typical hoof with a wound treatment 12 and a gauze cover 14. FIG. 3 shows the interdigital placement of the bandage wrap 16 of the invention and its tail 18. FIG. 4 shows the bandage 16 first wrap of the circumference of the cow's foot. FIG. 5 shows a right and left interdigital wrap 20, 22. FIG. 6 shows repetition of the wrapping steps demonstrated in FIGS. 5 and 6 to provide the final wrap 24 of FIG. 7.

To summarize the wrap procedure, the initial portion of the wrap 16 should be placed between the toes. Following, the wrap should be attached around the leg to allow after proper placement of the topical treatment. Several passages in between the toes say 2-3, should be performed next to increase the attachment of the treatment to the skin. Lastly, 1-2 turns around the leg facilitates the final adherence of the wrap 24 as well as the time to disintegration. Once cut, the last portion should be wet to allow the bandage to self-adhere. Any source of water or moisture could be used to facilitate the attachment. About 3 meters of bandage might be needed to achieve a proper wrapping procedure in the ordinary course. The prototype was thirty (30) microns thick.

Initially, 21 animals from two different farms were treated and evaluated for the usability of the dissolvable wrap. All animals were Holstein cows of different lactation periods, from young animals to lactating cows of 1^{st} to 3^{rd} lactation. All the animals presented an active digital dermatitis lesion at the moment of treatment, with an ulcer larger than 2 cm, in either the heel or the interdigital space. All treatments were applied by placing between 2-6 gr. of an antimicrobial compound under a cellulose gauze. Right after, the biodegradable wrap was applied following the description shown in FIGS. 1-7. The animals were monitored during the 2 hours post bandage and at fixed intervals during the day. An average of 1.8 days was observed for the duration of properly placed wrap. The time shown with a proper placement ranged between 2 hours and 10 days, preferably 1-5 days. No pressure wounds were observed in any case. In all cases, the attachment was effective when the tail end of the wrap was wetted with water.

In these tests the wrap was 35% starch by weight, and 50% PVA by weight, and 15% by weight colorant and plasticizer.

The table below shows the duration of the bandage before dissipation by self-dissolving and/or removal.

TABLE

| | | Bandage duration | |
|---|---|---|---|
| | | with appropriate | |
| Animal ID | Site Application | placement (days) | |
| 7203a | Leg | 0.09 | days (2 hours) |
| 7203b | Leg | 0.25 | days (6 hours) |
| 8187a | Leg | 3 | days |
| 8187b | Leg | 4 | days |
| 8215a | Leg | 3 | days |
| 8215b | Leg | 10 | days |
| 3434a | Leg | 0.25 | days (6 hours) |
| 3434b | Leg | 2 | days |
| 0873 | Leg | 0.12 | days (3 hours) |
| 7218 | Leg | 3 | days |
| 0925 | Leg | 4 | days |
| 7201 | Leg | 2 | days |
| 0859 | Leg | 1 | day |

The initial use tests showed several important points. The bandage needs to be stored in a dry place. The bandage can be successfully secured by simply applying water to the tail end of the posterior attachment. The treatment such as medicament liquid, pastes, powders, etc. should be separated from the wrap as illustrated here by gauze.

The new bandage is completely biodegradable and with zero accumulation of residues. Since water is a natural solvent of the wrap, moisture determines the rate of dissolution, especially in a high moisture environment. Finally, the new bandage is not elastic, and this did eliminate the risk of pressure wounds.

The preferred thickness is 40 microns to 80 microns.

What is claimed is:

1. A biodegradable polyvinyl alcohol (PVA) based bandage wrap, comprising: from about 50% to 100% by weight polyvinyl alcohol, and if less than 100% by weight, from about 20% by weight to 40% by weight of a copolymer selected from the group of starch copolymers and cellulosic copolymers, and from about 5% to 20% by weight of minors selected from the group consisting of bioactives, pharmaceuticals, deodorants, disinfectants, colorants, dispersal clays and bandage integrity modifiers and plasticizers.

2. The bandage of claim 1 which is 20% by weight to 40% by weight copolymer.

3. The bandage of claim 2 which is 20% by weight to 40% by weight starch copolymer.

4. The bandage of claim 3 which is 35% starch copolymers.

5. The bandage of claim 2 which is 20% by weight to 40% by weight cellulosic copolymer.

6. The composition of claim 1 wherein the weight ratio of PVA to starch copolymer is within the range of 6:1 to 1:1 to control dissolving time for the bandage wrap to from 2.0 hours to 10 days.

7. The composition of claim 1 wherein the weight ratio of PVA to starch copolymer is within the range of 3:1 to 1:1 to control dissolving home for the bandage wrap to from one day to 5 days.

8. The bandage wrap of claim 1 which is extruded to a bandage roll with a width within the range of 4 cm to 50 cm.

9. The bandage wrap of claim 1 which has a wrap thickness of from 20 microns to 200 microns.

10. The bandage wrap of claim 9 which has a wrap thickness of from 40 microns to 80 microns.

11. The bandage wrap of claim 1 wherein the bandage is 35% by weight corn starch copolymer.

12. The bandage of claim 1 wherein the starch copolymer: PVA copolymer weight ratio is adjusted to provide a bandage that will self-dissolve within a period of from 2.6 hours to 4.0 day, as desired.

13. A method of controlling the rate of dissolution of a self-disintegrating non-pressure bandage wrap, comprising:
adjusting the weight ratio of a PVA to starch copolymer of the bandage wrap to within the range of 6:1 to 1:1 to fix the disintegration rate of the bandage to within 2.0 hours to 10 days.

14. The method of claim 12 wherein the ratio of PVA to starch copolymer of the bandage wrap is within the range of 3:1 to 1:1 to control the disintegration rate to within 1 day to 5 days.
